(19) United States
(12) Patent Application Publication
(10) Pub. No.: US 2005/0158847 A1
(43) Pub. Date: Jul. 21, 2005

(54) Centrifugal array processing device

(76) Inventors: Stephen W. Fosdick, Medfield, MA (US); David M. Evans, N. Potomac, MD (US)

Correspondence Address: JOHN E. TOUPAL, 116 CONCORD STREET, FRAMINGHAM, MA, 01701, US

(21) Appl. No.: 10/499,051

(22) PCT Filed: Dec. 18, 2002
(86) PCT No.: PCT/US02/40322

Related U.S. Application Data
(60) Provisional application No. 60/342,697, filed on Dec. 20, 2001.

(51) Int. Cl. C12M 1/34

(52) U.S. Cl. 435/287.2; 435/288.5; 435/809; 422/72

(57) ABSTRACT

A device is provided in which a carousel is provided for use in such biochemical and chemical processes as polymerase chain reactions, using the centrifugal force resulting from rotation of the carousel to motivate fluid movement through microchannels embedded in microslides. The slides are arranged and positioned to function as fan blades vanes during rotation, to cause thermal regulation airflow through the device. Various other features are enabled by the present invention that improve efficiency and simplify processes such as those described.

### FIELD OF THE INVENTION

This invention relates to methods and apparatuses for driving biochemical and chemical reactions such as polymerase chain reactions in biological microsamples.

### BACKGROUND OF THE INVENTION

Numerous mechanical and automated fluid handling systems and instruments are known in the field of medical, biological and chemical assaying. For instance, U.S. Pat. No. 6,063,589 discloses a device wherein a disc-shaped master plate is divided into segments and each segment is adapted to provide a platform for driving a biochemical process through the effects of centrifugal force.

One drawback in such prior art apparatuses has been the difficulty in precisely and instantaneously controlling the sample flow and temperature during such a process. Designing systems for moving fluids on microslides through channels and reservoirs having diameters in the 10-100 um range and for controlling temperatures of those fluids to the degree of accuracy required for proper results has proven to be a daunting task. Microfluidic systems require precise and accurate control of fluid flow and temperature to control chemical reactions such as polymerase chain reactions (PCR's). Conventional heating and cooling mechanisms have been difficult to incorporate into microscale structures due to inherent conflicts-of-scale, and have generally provided less then desirable results.

Systems that use centrifugal force to effect fluid movement in microstructures, such as that disclosed in U.S. Pat. No. 6,063,589, address the need for a pumping mechanism to effect fluid flow, but does not address the problems associated with temperature control of the sample and do not provide a mechanism by which flow can be reversed and repeated. Total control of the sample flow is therefore not realized in such prior art systems. Furthermore, sample fluid flow and mixing should improve the hybridization of mRNA to DNA or oligonucleotide arrays on glass slides and provide more uniform results.

There remains therefore a need for a simple, reliable, economical and instantaneously-acting device and method for regulating the sample temperature and flow during the PCR process and/or the array hybridization process.

### OBJECTS OF THE INVENTION

It is therefore an object of the present invention to provide an improved system and method for driving polymerase chain reactions. Furthermore, it is an object of the current invention to provide an improved method for hybridization of samples to immobilized microarrays (either of DNA (as oligonucleotides), peptide nucleic acids or of peptides or proteins (e.g. antibodies, enzymes or other poly-amino acid molecules)).

It is a further object to provide such a system and method that improves temperature control during these reactions.

It is a further object to provide such a system that allows total flow control during the reaction.

It is a further object to provide such flow control including the ability to reverse and repeat the flow of sample across the microslide.

It is a further object to provide such temperature control including the ability to vary temperature in a controlled manner within the confines of an individual slide at any given time, and to vary overall slide temperature concurrent with the reversal and repetition of the sample flow.

Further objects and advantages of the present invention will become apparent to those of ordinary skill in the art upon review of the following disclosure thereof.

### SUMMARY OF THE INVENTION

The present invention provides a series of microslides, each tipped to lie at an angle from horizontal and stationed as spokes around a rotatable wheel such that a fan blade is formed with the slides serving as blade vanes. A motor, in communication with the wheel, is provided to rotate the wheel, thereby utilizing centrifugal forces resulting from the rotation to motivate movement of biochemical fluids through microchannels embedded in the microslides. Planar resistive heating elements (or those using Peltier heating methods) in communication with the slides are provided to heat the fluids in a controllable fashion. The rotation, together with fan blade configuration, also serves to move air through the system and to pass it over the microslides to increase the rate of cooling possible. Various microchannel configurations are provided according to the procedure being conducted.

Each heating element is connected to a programmable temperature control.

Heating of the fluids in each slide can either be controlled individually or, alternatively, as a group. Cooling of fluids in the slides is accelerated by the airflow passing thereover during rotation so that the reaction temperature can be adjusted instantaneously. The centrifugal forces on the liquid can be controlled according to the rotational speed of the wheel and the arrangement of the microchannels on the slides. Flow can be caused to reverse when the rotation is slowed since capillary action can overcome the centrifugal force and thereby allow total flow control.

Additional objects and advantages of the present invention will become evident from the following more detailed description and drawings of the preferred embodiment thereof.

### DESCRIPTION OF THE DRAWINGS

These and other objects and features of the invention will become more apparent upon a perusal of the following description taken in conjunction with the accompanying drawings wherein:

FIG. 1A is a sketch of a device for driving biochemical and chemical reactions in accordance with the preferred embodiment of the invention. It shows just two of the blades for clarity;

FIG. 1B is a sketch of a single blade assembly showing the end view of the heater and slide assembly;

FIG. 2 is a sketch of a slide assembly with reservoir and sample addition port (single large array area may contain microspots of immobilized biological materials attached to the substrate surface by chemical bonds). Arrays could be spots of DNA, PNA or peptides/proteins;

FIG. 3 is a sketch of the plan view of a slide assembly showing multiple addition reservoirs and a single waste reservoir. Liquids can be added to the reagent addition reservoirs and passed over an array of samples immobilized in the “array area”. By increasing centrifugal force, the liquid can be forced into the waste reservoir and can then be removed by aspiration (using a syringe assembly or pipette device);

FIG. 4 is a sketch of the plan view of a slide assembly showing two reagent addition reservoirs, a single waste reservoir joined by a serpentine capillary channel. The channel may contain immobilized material used to capture other species from the solution passing through the channel;

FIG. 5 is a sketch of a dual chamber array processing slide. Samples are added via ports (A, D, F, or G). By gravity, the liquid passes through the orifice at B, C, or E and impacts the array in the lower chamber. By centrifugation, the liquid will be forced towards the orifice “C” and will be forced up into the upper chamber of the slide (A,B,C). Relaxation of the centrifugal force will allow liquid back into array vessel (lower chamber). This allows rapid mixing of the solution. Also, if there were a heater below the array area this could be used to heat up the sample while rotor was stationary. When it was needed to cool the sample, the slide would be centrifuged and the liquid would pass to the upper chamber of the slide which is not heated. The air flow over this upper chamber would cool the liquid and lowering the rotation speed would allow this cooler liquid back into the lower chamber. The liquid between the two reservoirs would be mixed. Furthermore, liquid could be introduced through the ports or sample reservoirs (A,D,F, and G) and removed through the ports (A,D,F,G);

FIG. 6 is a sketch of a slide assembly that can be used to perform PCR reactions on a sample in a capillary. It consists of a sample reservoir and a waste reservoir in connection with a serpentine capillary channel. The channel is arranged so that it traverses the slide at an angle. The length of the channel across the slide is increased as it progresses towards the external edge of the slide. The channel is constructed to restrict flow by capillary action but to allow flow towards the outer edge of the slide under centrifugal force. As the liquid migrates along the channel it is exposed to the heat emanating from the heater below the surface. The heater is narrower towards the center of rotation and wider towards the outer edge of the slide. The heater is not as wide as the length of the channels at each point along the slide and is preferably located towards the midline of the slide. Thus liquid introduced at the sample reservoir remains at this point and does not enter the channel until exposed to an increase in centrifugal force. The heater element is set such that, at the midpoint along the channel, the fluid in the channel can be heated to >95 C. The fluid will flow along the channel at a speed determined by centrifugal force. As the fluid passes over the heater element it will be heated up and, as the channels extend beyond the heater element, the fluid will cool down. By varying the length of the capillary, the angle of the capillaries across the slide, the dimensions of the heater element and the speed of rotation, we can vary the temperature gradient that the sample sees during its path through the channel. Thus repetitive cycles of heating and cooling can be introduced to the sample. Since centrifugal force increases with distance from the center of rotation, the force seen by the sample will increase as it migrates further along each channel. In order to maintain a constant time for the sample in the appropriate heating zones, the length of the path that the liquid takes traversing the slide increases as the liquid migrates towards the outer edge of rotation. A cross-sectional view of the expected temperature at each point in the capillary channel shown in FIG. 7;

FIG. 7 is a sketch of a cross sectional graph of temperature in the capillary channel shown in FIG. 6. Since the heater element is located along the center of the slide, as the liquid passes over the heater it is heated (e.g. to 95 C). As the liquid moves further along the capillary it gets further away from the heater and is cooled. Cooling can be adjusted by the centrifugal force and the air flow over the slide surface as well as by the length of the capillary away from the heater element. Thus temperatures can be adjusted from room temperature (ambient) to the highest temperature required (e.g.˜95 C). Such temperature cycling is an integral part of the PCR process and hence the number of cycles can be adjusted by the number of passes that the capillary channel makes over the central heater core. Typically, the number of cycles required would be in the range of 10-50 and preferably 30-40;

FIG. 8A is a sketch of a multi-chamber device that allows multiple samples to be processed on a single slide. The spacing between the reagent addition ports allows direct liquid transfer from microtitre plates to the slide. Samples can also be aspirated from the same port used for sample addition and hence the product of the reactions can be collected. Liquid added to the sample port will flow down and settle at the bottom of the enclosed channel. The narrow opening at the top of the channel minimizes fluid loss due to evaporation. Centrifugation forces the liquid to the outside of each sample chamber and allows the liquid to come into contact with any materials in the outer capillary channel. Heating elements located below these outer channels can be used as elsewhere in this disclosure to heat the samples. Cooling can be effected by turning off power to the heaters and increasing the speed of rotation of the fan blade assembly to force a higher air flow over the slide surface. By passing light through the chamber (while stationary over the stationary light source or while rotating over it), a dye such as Sybr green or ethidium bromide can be used to determine how much duplex DNA material has been made during the thermocycling process since the latter only emits fluorescence when in the presence of the duplex strands of DNA and not in the presence of single stranded starting material;

FIG. 8B is a sketch of an adaptation of FIG. 8A to allow sample aspiration. This slide contains a series of capillaries that extend below the bottom edge of the slide. These capillary tubes are joined to one another within the slide area and this channel is coupled to a syringe assembly located at the center of rotation of the rotor assembly. The capillary tubes may be brought in contact with liquid samples within the wells of microtitre plates (for example) by raising the latter until the tubes are within each well. At this point, a small aliquot of sample is withdrawn into each capillary by withdrawing the barrel of the syringe. This produces an equal negative pressure within each capillary tube and liquid is lifted into the channels within the slide; and

FIG. 9 is a sketch of a multiple capillary processing slide. Each capillary channel can be accessed by a sample addition port located towards the center of rotation. These are located at a distance apart such that a multi-channel pipette can aspirate samples from a microtitre plate and dispense directly to the sample ports (typically this would require the ports to be at distances apart in multiples of 2.25 mm). Samples would be added to the ports, centrifugation would move the liquid into the capillary and heater elements below the capillaries would heat the samples. Samples could be cooled by centrifugal air flow over the slide. Samples could contain Reverse Transcriptase, nucleotide bases, fluorescent labeled bases, DNA polymerase or other enzymes to effect synthesis or degradation of oligonucleotides, polyamino acid molecules or other organic molecules.

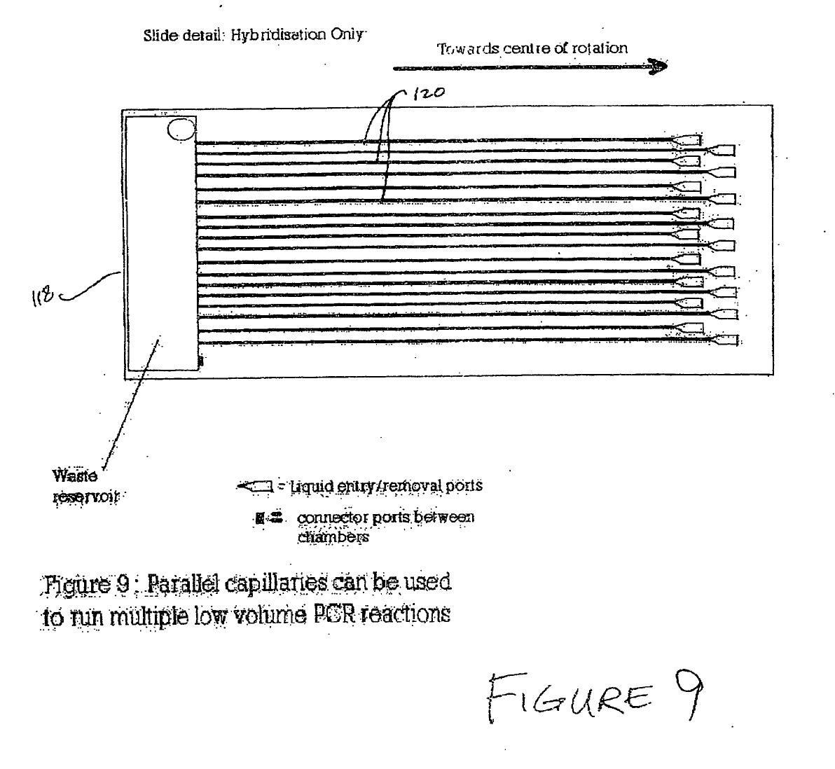

### DETAILED DESCRIPTION OF THE PREFERRED EMBODIMENT

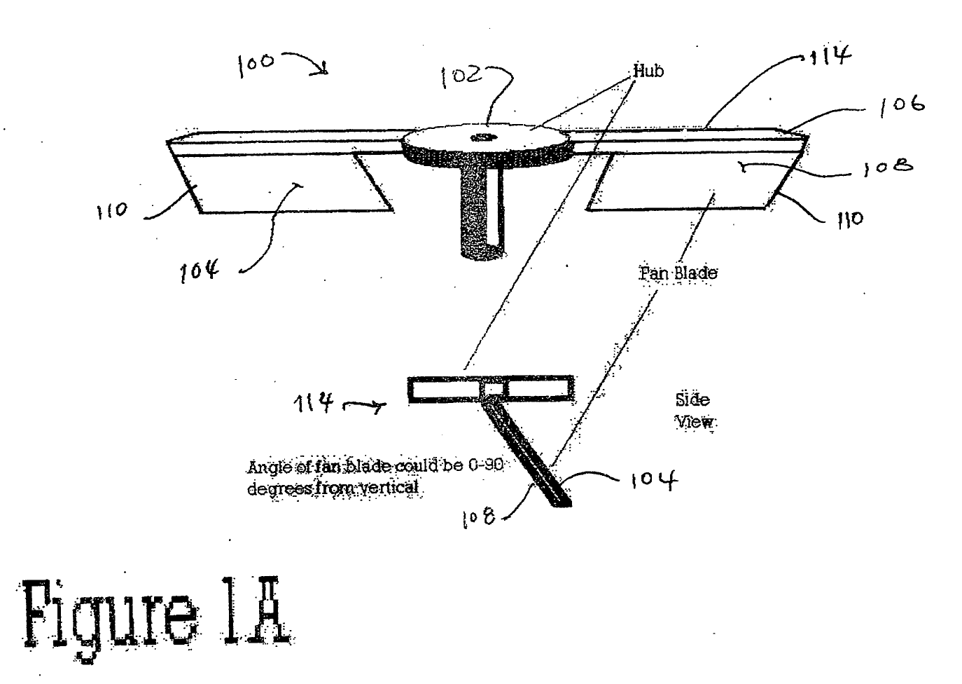

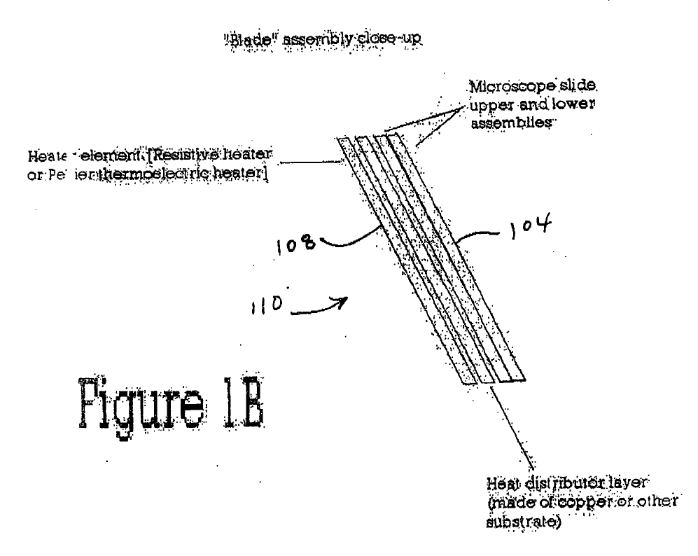

With reference made to FIGS. 1A through 8, the following describes a centrifugal microassay device according to the preferred embodiment of the invention.

As shown in FIG. 1A, the device 100 consists of a rotary drive rotor assembly 102 being able to locate fluid handling slides 104 or other flat surfaces into a carousel 106. Each glass slide 104 has upper and lower layers with engaged surfaces and is in contact with a heat exchanger heating (and/or cooling) element 108 as best shown in FIGS. 1A and 1B. The slide 104 and heater 108 form an assembly 110 which is at an angle of up to ninety degrees from horizontal. This effectively makes the carousel into a fan blade assembly 114.

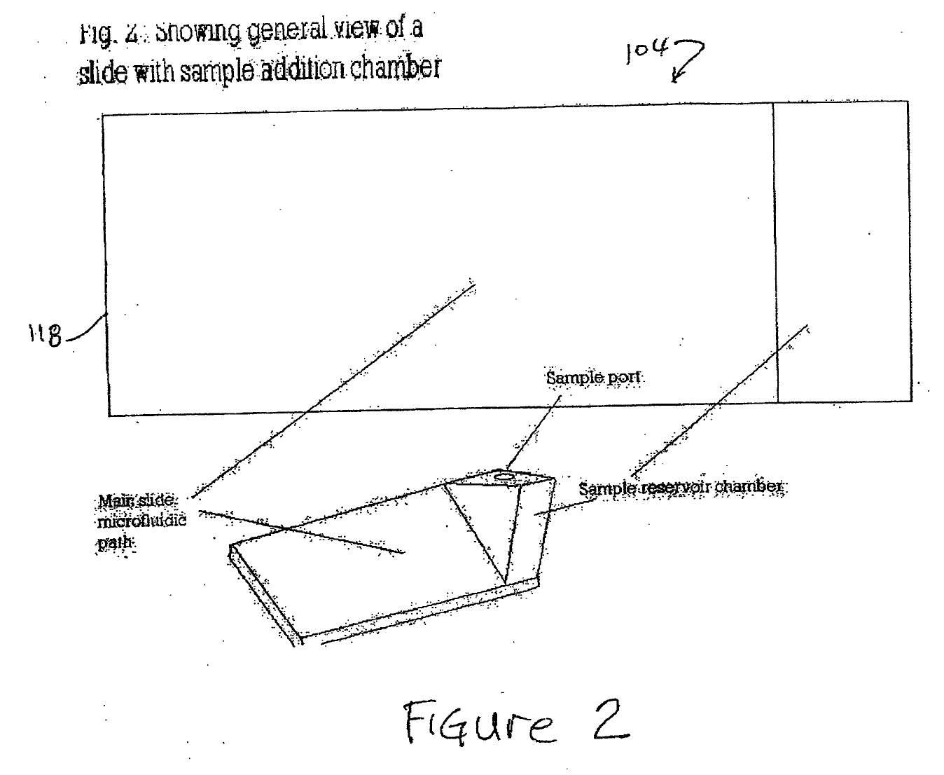

As shown in FIG. 2, the slides 104 are adapted to accept and contain microvolumes of fluids 1 16, which are added towards the center of rotation of the carousel 106 and, by spinning the carousel 106, and thus the slides 104, the fluids 116 circulate to the external edge 118 of each slide 104 through microchannels 120 engraved on the slide. The heating elements 108 would then be energized to heat the fluids 116 within the microchannels 120 during a heating phase. The temperature of the slide is monitored, such as through infrared observation of direct thermal sensing, and if the temperature exceeds a preselected “set” temperature, heating energy is reduced and centrifugation-forced air flowing over the slide surface provides an instantaneous cooling action.

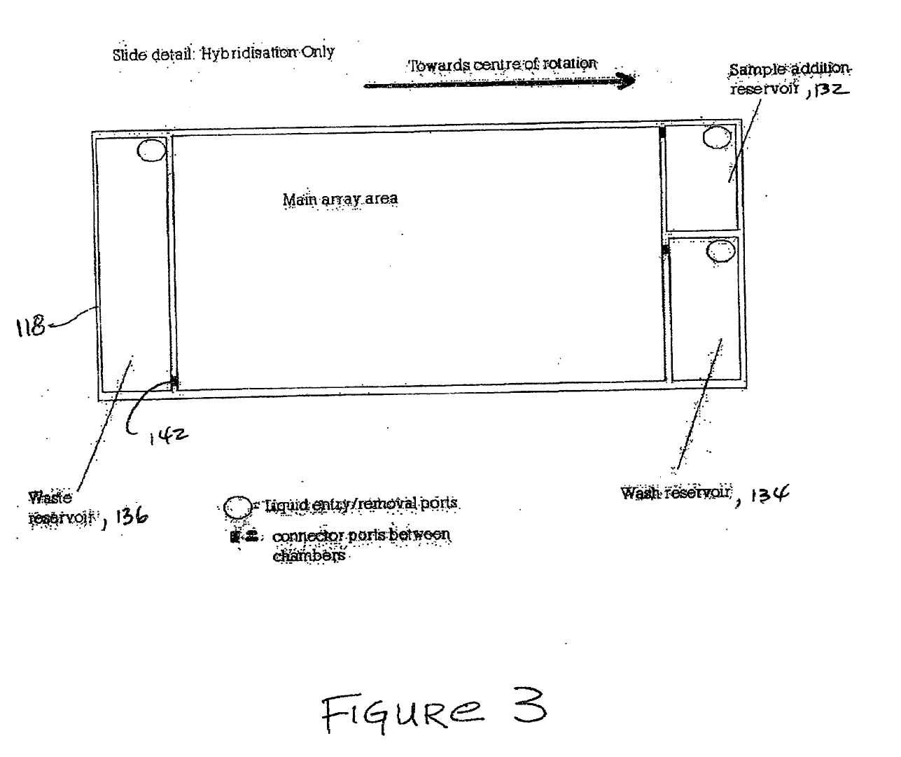

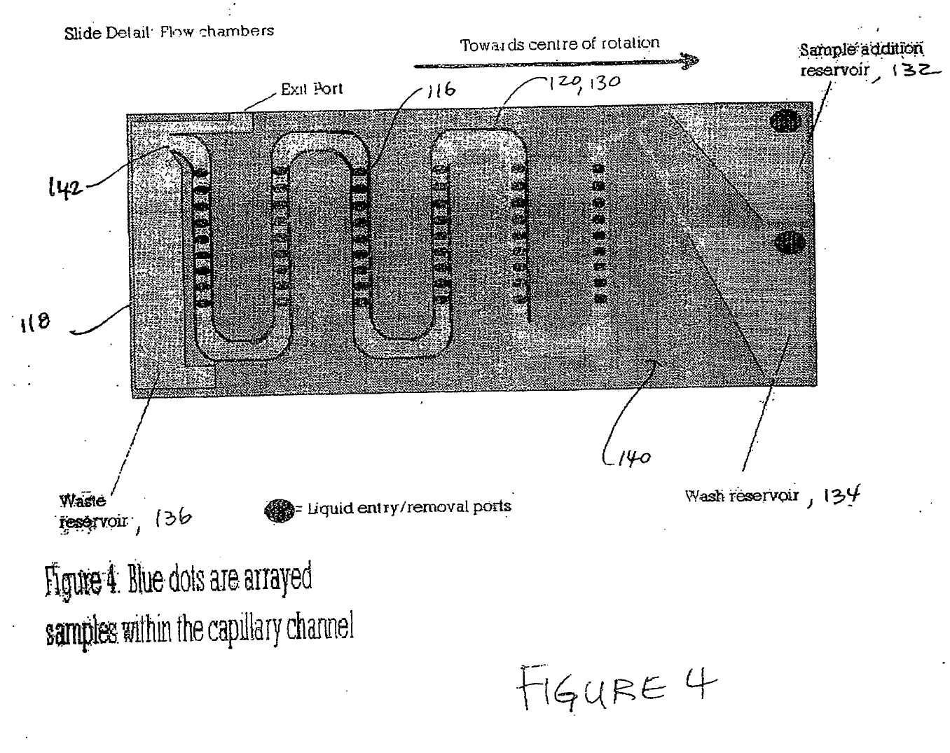

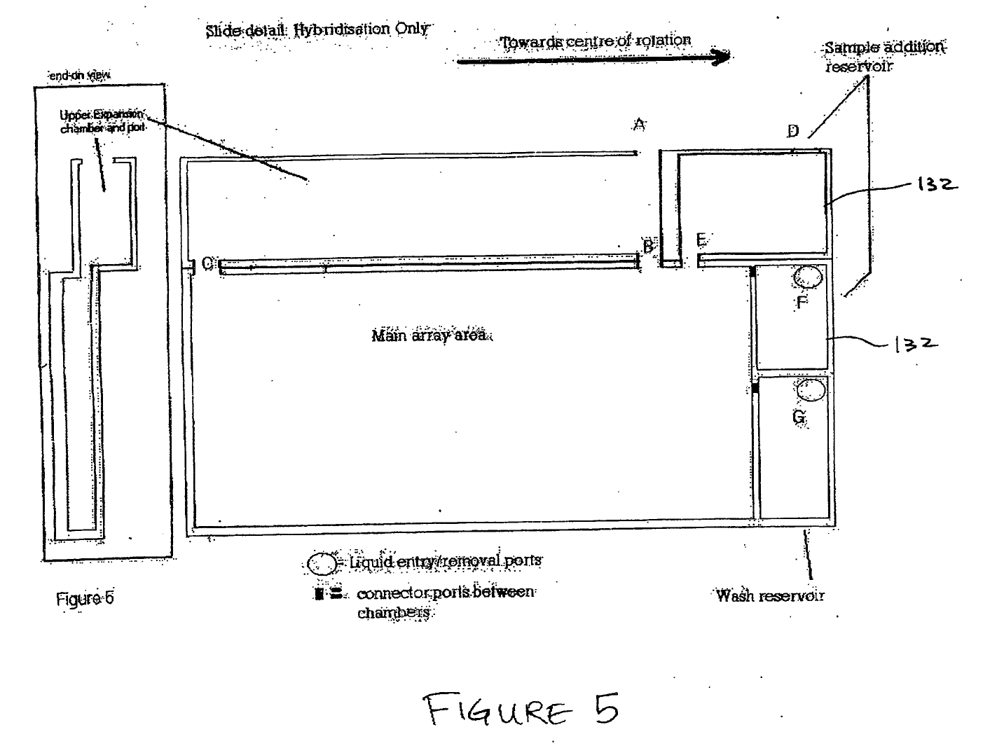

There are several phases of implementation of the device. These are:
1. A hybridization station where samples are introduced manually and temperature is kept constant at ˜55-75° C. Slides 104B are used as shown in FIG. 3.
2. A PCR adaptation for running PCR directly on the arrays. This involves cycling the temperature on the slides form ˜55° C. to ˜95° C. Slides 104B through 104E are used as shown in FIGS. 3 through 6.
3. A plastic liquid transfer device is used in cooperation with the slide. This comprises of a series of serpentine channels 130 molded in the surface of the device. Small drops of fluid in the order of 300 um in diameter, or less, are deposited into these channels. When the liquid channel surface is held against the array surface, the channels 130 are fully enclosed and the arrayed sample locations are located within each channel. These channels interface with a number of fluid supply reservoirs 132 and a wash reservoir 134 at source ends and interface with a waste reservoir 136 at opposite ends, as shown in FIG. 4. The supply reservoirs 132 and the wash reservoirs 134 are located at the end of the slide that is located closest to the center of rotation such that, at a given speed, the fluid from these reservoirs is fed into the array surface 140 or the serpentine channels 130. The waste reservoir 136 is located at the outer perimeter of the slide assembly and has an orifice 142 that, at slow speeds of rotation, is resistant to a flow of the fluid into the waste reservoir. At higher speeds, the fluid within the channels is forced into the waste reservoir 136. When the centrifugal force is removed, the capillary action of the channels pulls the liquid back into the channels 130 (or the array surface as shown in FIG. 5) and hence a mixing effect is produced. This is important in ensuring consistent quality of hybridization reactions as above.
4. The channels in one of the two opposing surfaces is located perpendicular to the axis of rotation and provides an extended light path for being able to monitor fluorescent or chemiluminescent reactions. By appropriate location of the excitation light and the detectors, the output from these points is monitored by rotating the slides (1 at a time or all at once) over the source and detector pairs corresponding to these light path locations within the slide.
5. An adaptation of 1 and 2 where there is an interface to the outside world that allows for the device to interface with standard microtitre plates, e.g. 96-well or 384-well plates. This allows the device to aspirate small volumes of samples that are then processed through the thermal cycle steps. In addition, it is feasible to simultaneously monitor for increases in the amount of sample being generated by measuring, either absorbance changes or fluorescence changes in the presence of an intercalating dye (e.g. Sybr green—which fluoresces only when double stranded DNA is present)

For performing hybridization reactions, the basic device 100 includes cartridges 132 that are adapted to contain a DNA or protein/peptide array imprinted on one of the mating surfaces, as shown in FIG. 3. The two portions of the cartridge are sealed by bringing them together. These cartridges are then placed onto the centrifugal device for processing (hybridization). As described above, the slides 104 serve as “fan blades” projecting outwards from the center of the cylinder. Each fan blade is angled relative to the horizontal plane. Below the fan blades, and in thermal communication therewith, is a heat exchanger heater element capable of raising the temperature within the cartridge 132 to ˜100° C. Temperature within the cartridge is monitored either by a simple thermocouple that is inserted into or attached to one of the fan blades or alternatively, using an IR thermal sensor (which allows non-contact detection of the temperature).

The method of operation is best understood with reference to FIG. 5. The cartridges, containing the samples of fluids to be hybridized, are first inserted into the carousel 106 and attached such that they are held in the aforementioned fan blade configuration. The device is sealed by closing the lid, and the temperature within the device is raised to an appropriate level by energizing a main heating element. It is desirable in these assays to maintain a constant temperature at all locations being processed to minimize the differences in results obtained from each sample. This would be possible in the current device since the fan blades would rotate allowing heated air from the main heater element to be drawn up over the cartridges. Furthermore, it has been observed that hybridization is improved if the sample solution being passed over the array is “mixed” or stirred while hybridization is ongoing. This is achieved because the cartridges are not fully filled with fluid and the chambers within are arranged and dimensioned to allow capillary action or “wicking” of the fluid towards the center of rotation of the carousel. The mixing of the fluid thus occurs as follows:
1. The carousel is rotated, hence drawing air from the heater element over the samples to ensure a more constant temperature within each.
2. The speed of rotation is increased, to increase the centrifugal force on the fluid within the chambers to force it towards the outer circumference of the carousel. After the fluid is forced to the outer edge of the chamber, the speed of rotation is lowered so that the liquid undergoes capillary wicking back towards the center of rotation of the device. By performing this cycle during the hybridization reaction the liquid is oscillated back and forth over the array surface and hybridization efficiency is increased.
3. To increase the ability to change the fluid layer over the arrays and to allow the addition of other external reagents it is feasible to have a capillary tube that passes through the array substrate and is exposed to the inner part of the carousel such that it is attached to a syringe assembly that is oriented vertically within the center of rotation. The syringe capacity is large enough that it aspirates fluid from all arrays simultaneously. Fluid is not mixed within the syringe, since the tubing between the syringe and the array substrates has a volume larger than that used for the hybridization step. While centrifuging the samples at constant speed, the syringe is aspirated to draw a sample from the outer radial edge of the substrate. A multi-directional valve is switched, allowing the connection of the syringe tubing with the inner chamber of the substrate. The syringe dispenses the fluid into this chamber, which, under centrifugal force, allows the fluid to pass outwards towards the circumference of the carousel and hence mix the sample over the array.

It is an additional aspect of the invention to provide for running PCR directly on the arrays. This involves being able to cycle the temperature on the chips from ˜55° C; to ˜95° C. As above, the cartridges are first attached to the carousel as fan blades and these are exposed to centrifugal force. In PCR, it is necessary to run many cycles where the temperature is varied typically in several steps e.g. 55-65° C. (annealing step), 70-75° C. (an extension reaction where new nucleotides are incorporated) and 95° C. (a melting step where the duplex strands are melted apart). This is accomplished in the centrifugal carousel as follows:

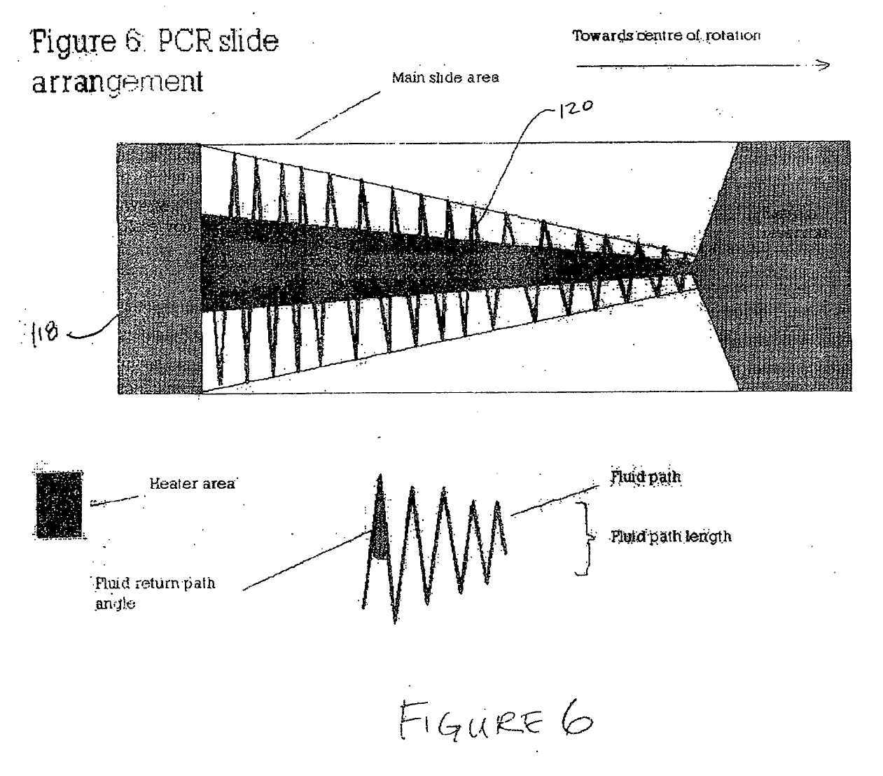

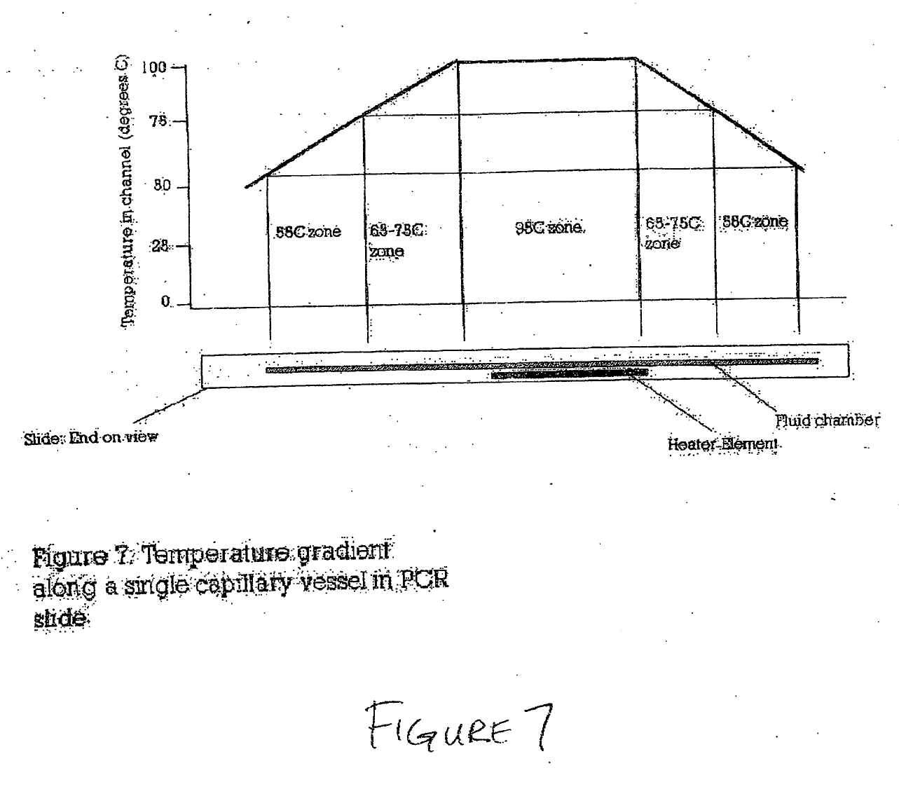

Taq polymerase is added to the samples along with the primer(s) and the nucleotide bases necessary for synthesis. As with the hybridization station above, the cartridges are rotated to allow for rapid heat transfer from the heater element up over the cartridges. The rotation ensures more thorough heat mixing and hence minimizes temperature variations within the different samples. In addition it allows for sample mixing as proposed above. A version of a slide that is used for this reaction is shown in FIG. 6 where it can be seen that a heater element runs along the center axis of the slide. A serpentine fluid path is located above the heater element and the path becomes longer as it progresses towards the outer perimeter of rotation of the slide. This extension of the path length is varied by increasing or decreasing the angle between successive channels across the face of the slide and/or by varying the path length of each successive cross channel. Thus, as fluid migrates towards the outer perimeter of the slide, it experiences an increase in centrifugal force. This force tends to accelerate the speed of migration of the fluid. Elongation of the path length and reduction of the angle between successive turns in the path, together with the accompanying widening of the heater area towards the outer perimeter, ensures that the fluid within the fluidic channel is exposed to the heat source for the same duration during each cycle, as seen in the graph of temperature vs. position within a channel shown in FIG. 7.

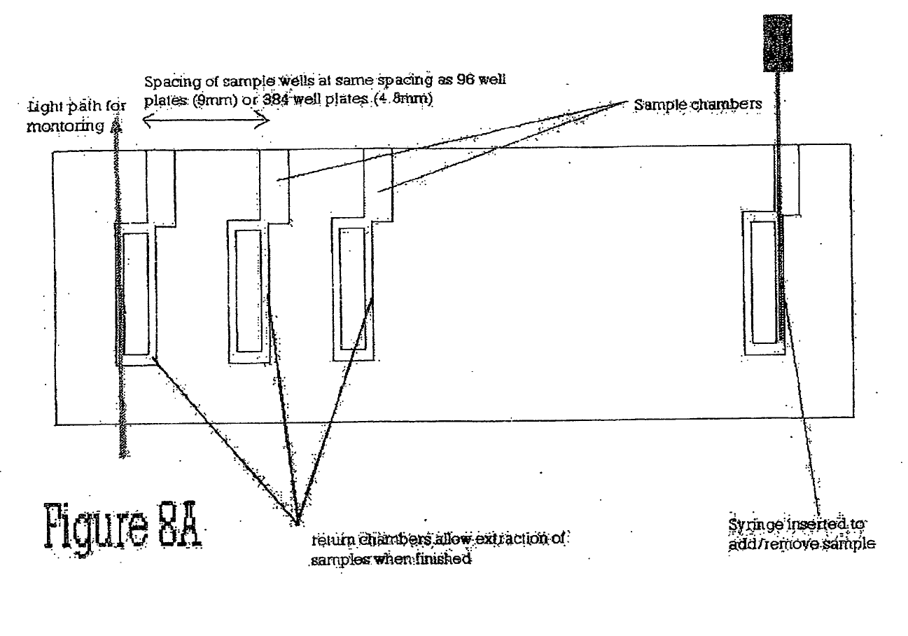

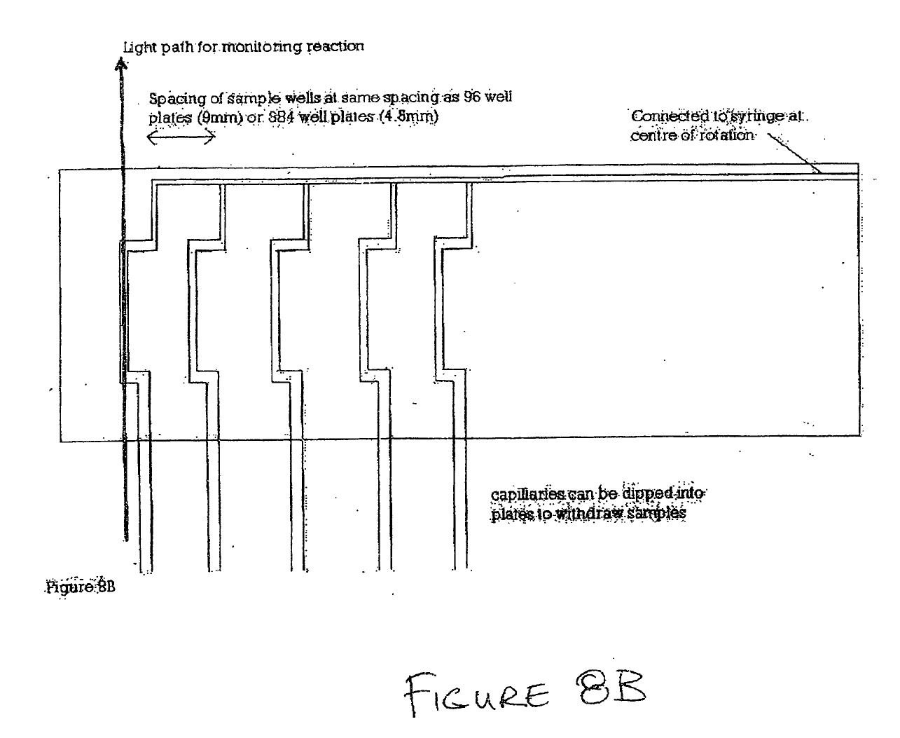

In a Real Time PCR (RTPCR), the PCR amplification reaction is monitored after every cycle to determine the amount of probe that has been generated during the reactions. Typically this is accomplished by inclusion of an intercalating dye that binds between the two strands of the duplex formed during the extension reaction. Thus the amount of fluorescent signal generated is proportional to the amount of the duplex formed; the number of cycles required to generate a given amount of fluorescent signal is related to the initial amount of a sample of interest that was present. It is increasingly important to be able to quantitate the amount of DNA in a smaller volume. The current methods require a 25 ul sample. One of the problems common in the prior art is that the detection of the fluorescent signal generated in the well of a typical PCR plate requires illuminating a large area. Consequently the effective light path for signal generation is very small. The present invention allows smaller sample volumes to be used and maintains a long path length for quantification of the fluorescent signal. In addition, the present invention enables aspiration of a small aliquot (2-5 ul) from a microtitre plate for performing the reaction. Centrifugal heat dispersion of the fan blade assembly is employed to control the sample temperature. In addition to the cartridge described above there are various arrangements that can be used for aspirating fluid into the PCR reaction capillaries:
1. Liquid is dispensed into a small sample well within a slide as shown in FIG. 8;
2. The syringe assembly described above is connected to individual capillary tips that protrude from the bottom of the cartridge such that they are immersed in fluid in the bottom of a microtitre plate as shown in FIG. 8B. The capillary tips are inserted into the wells of the plate by lowering the appropriate cartridge of the carousel while the carousel rotor is in the correct position. The syringe aspirates a volume such that each capillary draws up the volume needed. The liquid path is disposed within the plane of the cartridge but describes two right angle turns. This has a dual purpose, as shown in FIG. 8B, which is: (A) so that under centrifugal force the liquid is forced to fill the channel towards the outer circumference of the cartridge which minimizes the exposed cross sectional surface area of the liquid to diminish losses of samples by evaporation at the elevated temperatures necessary for RTPCR; and (13) so that the channel that the fluid enters has glass end caps on it such that light is passed along the length of the channel. This allows a light source below the cartridge to shine light along the length of the channel. The light exiting the top of the channel is filtered for the correct frequency of light for the emitted fluorescence and read using a detector cell.

An alternative method for aspirating the sample requires that each capillary path is associated with a single wire “plunger” that rides within the capillary channels above the light path channel. The wire plungers are joined in the radial direction of the carousel such that, by driving the joined pistons up or down the liquid in the microtitre plate is aspirated or dispensed. Again the offset in the light path channel allows fluorescence to be monitored.

In another version of this approach, the capillary tips that enter the well of the plate meet a block that contains the offset channels. Thus bringing the wires up while the capillary tips are immersed aspirates liquid up into the “reaction block”. A sliding mechanism slides the capillaries such that they are now over the light source for fluorescent measurement and so that the lower end of each capillary is “blocked”. The reaction block is heated either as described above or by a Peltier device or other heating element incorporated directly into the block It is still possible to measure the fluorescent signal during each cycle of the reaction. The block for forming the capillaries is made of glass that has been etched with the channels. This allows for passage of fluorescence through the block.

The invention provides novel means for heating and cooling the centrifugal devices. Although the examples above indicate that it is possible to heat the centrifugal devices using only a heating element below the Fan assembly, other methods of modulating temperature are also used. Using only a single main heating element could result in an amount of heat that could be very high in order to overcome the insulating property of the surrounding air. This could result in a very high temperature within the chamber in order to provide the necessary temperature within each chamber. To overcome this, other methods of heating the slides and their enclosed channel structures are provided, which may include:
A) Peltier devices. These devices take electrical current and produce heat on one side of the device and cooling effect on the opposite side. By direct apposition of the heating side of the Peltier device with each chamber there is a more efficient heat transfer to the chamber. This means that the overall temperature within the carousel chamber is not as high and results in a faster cooling of the individual slides, since the mass of heated surfaces is not as great as with that in a single main heating element design.
B) Inked Heating. InfraRed heat lamps can produce intense heat in objects without getting too hot themselves. Thus these units can be used to heat the slides. Upon turning off the heat source the chambers are cooled as above by spinning which draws air from outside the unit over the fan blades.
C) MicroWave Heating. Microwaves have the added advantage of being able to heat aqueous samples very quickly without impacting on the surrounding material. Thus, the microwave radiation is turned on and aimed at the carousel while it is spinning. The spinning results in an averaging effect of the time that each chamber was exposed to the radiation. This in turn results in a more even heating of all chambers. Since the material surrounding each chamber is made of a material that does not heat upon exposure to the microwave radiation, the heat mass can be, and is, very low. Thus, as the blades are spinning, the cooling effect is also very rapid as air is drawn over the blades. The only caveat with using microwaves is that the components used for the centrifugal device must be properly shielded if they contain exposed metal objects since they could otherwise interfere with the microwaves and could result in inefficient heating and/or sparks being generated.
D) Resistive heating elements. These elements are attached to the slide and use electrical energy to create heat. They are cooled by the airflow over them as the carousel is spinning. These units are attached to the slide surface or could be incorporated onto the inner side of one half of the slide assembly. This allows direct heat contact with the sample and further reduces the time needed to heat the sample since heat does not need to pass through an insulating layer of material.

Additional processes in which the present invention might be used include chemical synthesis in which repetitive additions, for example of nucleotide bases to a DNA strand or of amino acids to a peptide, are required, or in the synthesis of organic compounds, where there is a need to add materials and/or change reaction temperature.

It should also be understood that the foregoing disclosure emphasizes certain specific aspects of the invention and that all modifications or alternatives equivalent thereto are within the spirit and scope of the invention.

1. A fluid processing system comprising:
a frame;
a rotary drive assembly coupled to said frame and adapted to produce rotation thereof about a rotational axis;
a plurality of fluid handling members mounted on said frame and circumferentially spaced apart about said rotational axis, each of said members defining at least one channel forming a fluid flow path extending radially from said rotational axis;
fluid supply means for feeding fluid into said channels at source ends thereof adjacent to said rotational axis;
heat exchanger means for selectively controlling the temperature of said fluid fed into said channels;
speed control means for controlling the rotational speed of said rotary drive mechanism to generate centrifugal force causing said fluid to circulate through each said channel from said source end to an opposite end; and
receptacle means for receiving from said opposite ends said fluid circulating in said channels, and wherein said channels are shaped and arranged to produce fluid flow into said receptacle means in response to a given rotational speed of said rotary drive mechanism, and to prevent at a lesser rotational speed centrifugal force induced fluid flow into said receptacle means while creating capillary action producing fluid flow from said receptacle means into said channels and toward said source ends.

2. (canceled)

3. (canceled)

4. (canceled)

5. A fluid processing system according to claim 1 wherein each said fluid handling member is a slide.

6. A fluid processing system according to claim 5 wherein each said slide comprises upper and lower layers with engaged flat surfaces defining said channel.

7. A fluid processing system according to claim 6 wherein said channel is molded into one of said flat surfaces.

8. A fluid processing system according to claim 6 wherein said channel is engraved into one of said flat surfaces.

9. A fluid processing system according to claim 6 wherein said slides are transparent and said system comprises means for detecting fluorescent signals generated in said channels.

10. A fluid processing system according to claim 6 wherein each said channel comprises components extending perpendicular and parallel to said axis.

11. A fluid processing system according to claim 10 wherein said heat exchanger means comprises a heater element overlying each of said slides.

12. A fluid processing system according to claim 11 wherein each said channel is serpentine with legs increasing in length between said source end and said opposite end.

13. A fluid processing system according to claim 12 wherein said heat exchanger means comprises a heater mounted below said fluid handling members so as to provide heated air contacting and driven by said fluid handling members.

14. A processing system according to claim 13 including receptacle means for receiving from said opposite ends said fluid circulating in said channels.

15. A fluid process system according to claim 14 wherein said channels are shaped and arranged to produce fluid flow into said receptacle means in response to a given rotational speed of said rotary drive mechanism, and to prevent at a lesser rotational speed centrifugal force induced fluid flow into said receptacle means while creating capillary action producing fluid flow from said receptacle means into said channel and toward said source ends.

16. A fluid processing system according to claim 15 therein said fluid handling member is a slide.

17. A fluid processing system according to claim 16 wherein each said slide comprises upper and lower layers with engaged flat surfaces defining said channel.

18. A fluid processing system according to claim 5 wherein each said channel comprises components extending perpendicular and parallel to said axis.

19. A fluid processing system according to claim 22 wherein said heat exchanger means comprises a heater element overlying each of said members so as to provide conducive heating thereof.

20. A fluid processing system according to claim 5 wherein said fluid supply means comprises a supply reservoir mounted on each said slide and said receptacle means comprises a waste reservoir mounted on each said slide.

21. A fluid processing system comprising:
a frame;
a rotary drive assembly coupled to said frame and adapted to produce rotation thereof about a rotational axis;
a plurality of fluid handling members mounted on said frame and circumferentially spaced apart about said rotational axis, each of said members defining at least one channel forming a fluid flow path extending radially from said rotational axis, and wherein each said channel comprises components extending perpendicular and parallel to said axis;
fluid supply means for feeding fluid into said channels at source ends thereof adjacent to said rotational axis; and
heat exchanger means for selectively controlling the temperature of said fluid fed into said channels.

22. A fluid processing system comprising:
a frame;
a rotary drive assembly coupled to said frame and adapted to produce rotation thereof about a rotational axis;
a plurality of fluid handling members mounted on said frame and circumferentially spaced apart about said rotational axis, each of said members defining at least one channel forming a fluid flow path extending radially from said rotational axis and being separated so as to allow air flow therebetween;
fluid supply means for feeding fluid into said channels at source ends thereof adjacent to said rotational axis; and
heat exchanger means for selectively controlling the temperature of said fluid fed into said channels.
